(19) United States
(12) Patent Application Publication
(10) Pub. No.: US 2010/0191313 A1
(43) Pub. Date: Jul. 29, 2010

(54) APPARATUS FOR LIGHT THERAPHY AND METHOD FOR USE

(76) Inventor: CHARLES A CARR, Aberdeen, WA (US)

Correspondence Address: Karr Tuttle Campbell;ATTN: PRIYA SINHA CLOUTIER, 1201 Third Ave., Ste 2900, Seattle, WA, 98101, US

(21) Appl. No.: 12/361,688

(22) Filed: Jan. 29, 2009

(51) Int. Cl. A61N 5/06

(52) U.S. Cl. 607/90

(57) ABSTRACT

The purpose of the current invention is to provide an apparatus that provides light therapy, to those afflicted with SAD, which does not interfere with the user's day. The current invention can be used while working on a computer or watching television, for example. Additionally, the apparatus is portable so it can easily be stashed into a purse or briefcase, for example, so that the apparatus can be used in the office, home, or away location.

### CROSS-REFERENCES TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND

Seasonal affective disorder (“SAD”) is a mood disorder in which individuals experience depressive symptoms in winter months or in places where sunlight is at a premium, even in summer months. Individuals with SAD may feel depressed, sleep too much, have little energy, and crave sweets and starchy foods.

In the United States, Alaska has a SAD rate of up to 8.9%. (http://www.britebox-sad-lamps.com/sad-study-side-effects.php) Around 20% of Irish people are affected by SAD, according to a survey conducted in 2007. The survey also shows women are more likely to be affected by SAD than men.

(http://www.breakingnews.ie/ireland/mhmhojididid)

There are many types of treatments for SAD, including light therapies, medication, ionized-air administration, cognitive-behavioral therapy and carefully timed supplementation of the hormone melatonin. Light therapy treatment is used by most sufferers of SAD.

Light Therapy uses a specially designed lamp to provide much more intense illumination than traditional incandescent bulbs are capable of. The light is usually white “full spectrum”, although blue light is also used. The sufferer sits at prescribed distance, commonly 30-60 cm, in front of the lamp with her/his eyes open but not staring at the light source. Most treatments take 30-60 minute however, this may vary depending on the individual. Many patients use the light therapy in the morning because it is thought that morning light is superior to evening light.

However, because the treatment requires the sufferer to sit for an extended period of time facing a light, the treatment becomes tedious; even if effective, many SAD sufferers eventually discontinue treatments. One study has shown that up to 69% of patients find the treatment inconvenient and as many as 19% stop use because of this. (Avery, D H; Eder D N, Bolte M A, Hellekson C J, Dunner D L, Vitiello M V, Prinz P N (2001). “Dawn simulation and bright light in the treatment of SAD: a controlled study”. Biological Psychiatry 50 (3): 205-216)

The purpose of the current invention is to provide an apparatus that provides light therapy, to those afflicted with SAD, which does not interfere with the user's day. The current invention can be used while working on a computer, watching television, or using the stair master, amongst others. The apparatus is portable so it can easily be stashed into a purse or briefcase. Consequently, the apparatus can be used in the office, home, or away location.

### BRIEF SUMMARY OF THE INVENTION

The purpose of the current invention is to provide an apparatus that provides light therapy, to those afflicted with SAD, which does not interfere with the user's day.

### BRIEF DESCRIPTION OF THE SEVERAL VIEWS OF THE DRAWINGS

Other features and advantages of the present invention will become apparent in the following detailed descriptions of the preferred embodiment with reference to the accompanying drawings, of which:

FIG. 1 is an elevation view of the apparatus in a preferred embodiment positioned around a computer screen;

FIG. 2 is a side elevational view of a fixture;

FIG. 3 is a bottom elevational view of a fixture;

FIG. 4 is a top elevational view of a fixture;

FIG. 5 is an elevational view of one preferred means to hold the fixture in place on a horizontal surface;

FIG. 6 is an elevational view of another preferred means to hold the fixture in place on a horizontal surface.

### DETAILED DESCRIPTION OF THE INVENTION

The present invention is described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete and will fully convey the scope of the invention to those skilled in the art.

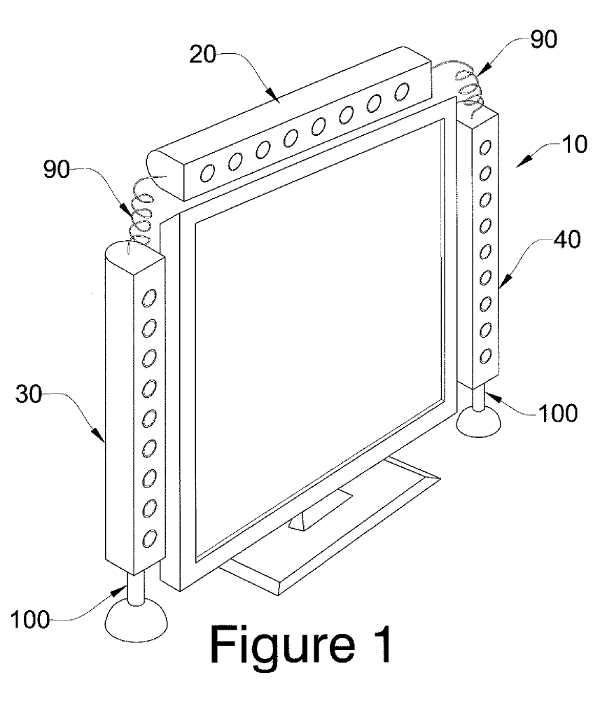

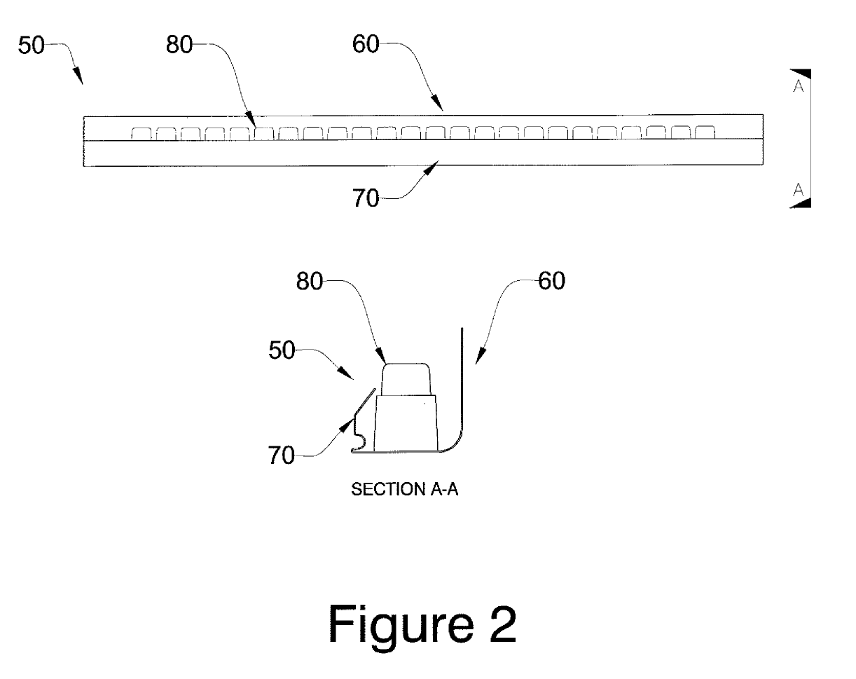

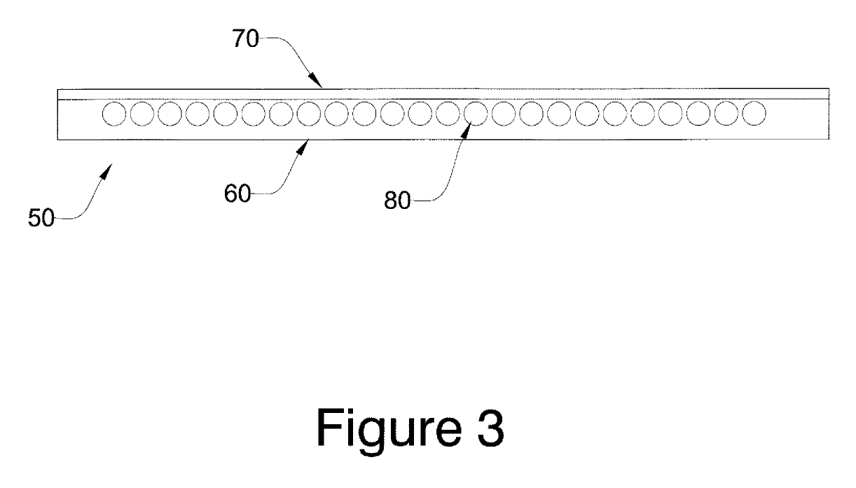

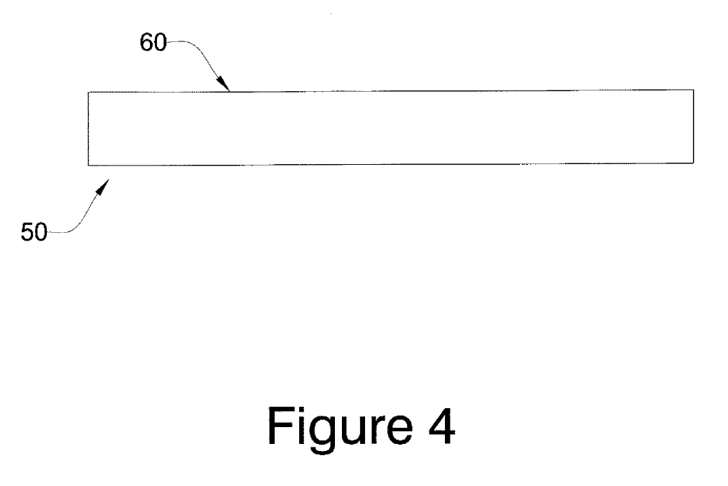

Referring to FIG. 1 through 4, the present invention is an apparatus (10) that provides light therapy for persons who suffer with SAD. The apparatus (10) comprises at least a first side (20), a second side (30), and a third side (40). Each side (20, 30,40) comprises a fixture (50); each fixture (50) has a visor side (60) and a bottom side (70). The visor side (60) extends over the bottom side (70). A band of lights (80) is attached to the bottom side (70) of each fixture (50). Preferably, the band of light (80) comprises LCD white lights which are rated at 5600 k or higher to simulate day light. The lights can be powered by any known or unknown power source.

The first side (20) is attached to the second side (30) and a third side (40) to form a “U” shape. The first side (20) is attached to the second side (30) by a means (90) that will allow the distance between the first side (20) and the second side (30) to increase or decrease as needed. The first side (20) is attached to the third side (40) by a means (90) that will allow the distance between the first side (20) and the third side (40) to increase or decrease as needed.

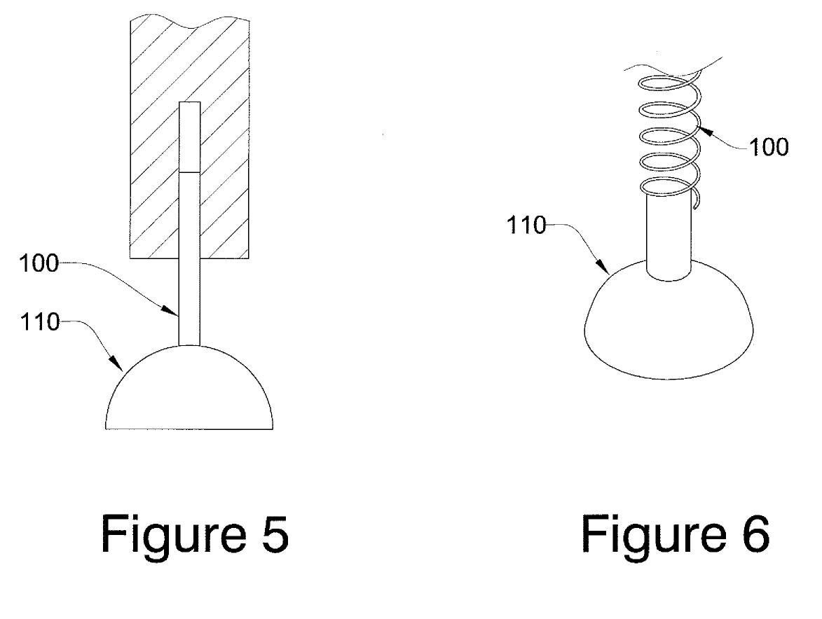

Referring to FIGS. 1, 5, and 6, in one embodiment, the second side (30) further comprises a means to adjust the distance (100) between the fixture (50) and a horizontal surface. The means to adjust the distance (100) is a telescoping arm. The telescoping arm comprises a foot (110) that would allow the side to lay flat on a horizontal surface.

In another preferred embodiment, the means to adjust the distance (100) is a spring-type material that can compress or expand. The spring-type material comprises a foot (110) that would allow the side to lay flat on a horizontal surface.

In another preferred embodiment, the fixtures (50) comprises a means to removable attach said fixtures (50) to a television or computer monitor, for example. Said means can be removable adhesive, a hook and loop system such as VELCRO™, amongst others.

The user of the apparatus (10) simply places it around his television, computer screen, CD player, or iPhone, amongst others, during regular activity. Consequently, he does not have to “waste” time looking into lights.

What is claimed:

1. The present invention is an apparatus that provides light therapy for persons who suffer with SAD.

2. The apparatus of claim 1 comprises at least a first side, a second side, and a third side; each side comprises a fixture.

3. Each fixture of claim 2 has a visor side and a bottom side.

4. The visor side of claim 3 extends over the bottom side of claim 3.

5. A band of lights is attached to the bottom side of claim 3 of each fixture.

6. The first side, second side, and third side of claim 2 are attached to form an “U” shape.

7. The apparatus of claim 1 is further comprises a means to place the apparatus around a television screen, computer screen, personal digital assistant screen, cell phone screen, amongst others.

8. The apparatus of claim 1 is further comprises a means to attach the apparatus around a television screen, computer screen, personal digital assistant screen, or a cell phone screen, amongst others.

9. A method to use the apparatus of claim 1 comprises:
(a) placing the apparatus around a television screen, computer screen, personal digital assistant screen, or a cell phone screen, amongst others;
(b) allowing power to lights;
(c) working, playing or watching the television, computer, personal digital assistant, or cell phone screen for treatment of SAD.
